(19) United States
(12) Patent Application Publication
(10) Pub. No.: US 2008/0167675 A1
(43) Pub. Date: Jul. 10, 2008

(54) System, Device And Method For Recording Pressure Profiles In The Pharynx And In The Upper Isophageal Sphincter Upon Swallowing

(76) Inventors: Sture Hogosta, Falun (SE); Mats Linden, Falun (SE)

Correspondence Address: YOUNG & THOMPSON, 209 Madison Street, Suite 500, ALEXANDRIA, VA, 22314, US

(21) Appl. No.: 11/883,295

(22) PCT Filed: Jan. 31, 2006
(86) PCT No.: PCT/SE06/00140

§ 371 Date: Jul. 30, 2007

(30) Foreign Application Priority Data
Feb. 3, 2005 (SE) 0500256-3

(51) Int. Cl. A61B 18/18; A61B 5/00

(52) U.S. Cl. 606/196; 600/301

(57) ABSTRACT

A system, device and method for recording a pressure profile from the pharynx and upper esophageal sphincter (UES) upon swallowing, includes a manometric catheter insertable into the esophagus and having a first lumen connected to a first pressure sensor and to a water supply by which the first lumen is continuously flushed with water in an open perfusion manometry system for registering the activity of the pharyngeal muscles, a second lumen connecting a water-filled, inelastic elongate balloon to a second pressure sensor in a closed system by which pressure variations in the upper esophageal sphincter is simultaneously and continuously registered, and a processor unit recording and displaying timely correlated pressure profiles derived from pressure data registered by the two pressure sensors. The balloon is filled with water to atmospheric pressure outside the esophagus, and insertable therein for registering absolute pressures from the UES during a normal swallowing act.

System, device and method for recording pressure profiles in the pharynx and in the upper esophageal sphincter upon swallowing.

### TECHNICAL FIELD

The present invention relates to a system, a device and a method for recording pressure profiles in the hypopharynx and upper esophageal sphincter, and more specifically to a system, device and method designed to register and display the coordination of muscular contraction and relaxation during a normal swallowing act.

### BACKGROUND

Patients with dysphagia and radiological evidence of upper esophageal dysfunction, e.g. a cricopharyngeal bar, may suffer from one of three main types of abnormalities—achalasia, spasm or hypertrophy of the cricopharyngeal muscle. Some of these patients may benefit from cricopharyngeal myotomia or other therapeutic measures. In selecting patients suitable for treatments it is of great value to determine the static and dynamic pressures in pharynx and the upper esophageal sphincter (UES), since there is frequently a discrepancy between radiographic and manometric findings. Manometry thus is necessary for correct diagnosis of swallowing disorders. When cricopharyngeal myotomia is performed on radiological criteria alone, the success rate may be as low as 50%. Traditional manometric methods have relied on perfused catheters or solid-state catheters. Though suitable for registering pressure variations in the middle and lower parts of esophagus, these methods have limitations when applied to the UES. The reasons include difficulties in assessing the rapid pressure wave traveling through the relatively short UES, and the pharyngo-laryngeal elevation during swallowing which may dislocate the manometric device. Further, the UES is not a sphincter with circumferentially equal pressure in all directions—in the anterior-posterior direction the pressures are much higher than in lateral direction. This is a factor that conventional manometric methods fail to recognize. In a sphincter with variable pressure profile a perfusion system will always record the lowermost existing pressure and a balloon will record the opposite, that is the highest, which is of most interest.

### PRIOR ART

Rubber balloon devices were used in early manometric studies, but their high compliance made the registration unreliable.

Conventional systems are usually based on perfusion manometry performed through multi-lumen catheters, sleeve-device catheters and solid-state catheters. Multi-lumen catheters perfuse water at different locations for registration of pressure waves, and are used for registration in corpus oesophagus and the lower sphincter. Their need to perfuse at a high rate to achieve low compliance has, among other things, made them unusable for UES registration. The sleeve-device catheter perfuse water under a thin membrane attached to the catheter and is used for registration of the UES. The sleeve-device catheter system can register continuously but register in the direction of the membrane only, and not circumferentially. Furthermore, as in all perfusion systems, the lowermost pressure rather than the highest is achieved. However, investigation of patients with problems to swallow should preferably include a continuous and simultaneous registration of pressure conditions in the regions of the pharynx as well as the upper esophageal sphincter.

Solid state catheters are clinically also used to measure the UES pressure profile. These are of different kinds and some even have circumferential registration capacity. They have low compliance and high frequency response rates. They are small cylindrical rods incorporated in plastic catheters. Their shortcoming is due to a relatively short length and during the elevation of the UES upon swallowing they can be dislodged from the pressure zone.

A manometry apparatus for measuring lower esophageal sphincter (LES) compliance is described in U.S. Pat. No. 6,773,452 (SHAKER). The probe consists of a first extendable member, such as a balloon, which is inflated inside the esophagus at a position above the LES in order to relax and trigger motility of the esophagus, thereby simulating a natural swallowing response that permits measurement, through a second extendable member such as a second balloon, the amount of resistance provided by the LES without the underlying tonic interference, the LES being ordinarily constricted. A third extendable member such as a third balloon located in the stomach secure the position of the second balloon in the LES. The device is thus not appropriate for measurement performed on the upper sphincter due to its construction and properties. A balloon of corresponding dimensions introduced in the hypopharynx will probably not be tolerated by most patients. Furthermore, the size of the balloon will distend the UES and create a raised muscular tonus. When introduced in the esophagus this known apparatus will most certainly obstruct a normal swallowing act, the Shaker apparatus being designed and implemented for recording pressures during a simulated swallowing act initiated by inflation of an upper extendable member. With respect to the recording of correct absolute pressures the elongate balloon must, before introduction into the esophagus, be filled with a fluid to atmospheric pressure. An overfilled inelastic balloon, i.e. filled to a higher pressure than atmospheric pressure, will markedly increase its sensitivity for pressure and temperature and false registrations will be obtained. Incomplete filling of the balloon will increase compliance. The probe suggested by Shaker is filled in the esophagus and thus atmospheric pressure cannot be achieved properly. The fluid must be water or some other incompressible fluid in order to respond to high pressure transients occurring in the UES. The device described by Shaker is thus suitable for lower esophageal sphincter measurements, but with respect to recording true pressures in the UES, the Shaker apparatus is less suitable. A man skilled in this art would not apply the Shaker probe or find therein the teachings for designing a method and apparatus suitable for performing true UES and hypopharyngeal pressure recordings during a normal swallowing act.

Further background may be found in. e.g., “Instrumentations and Methods for Intraluminal Esophageal Manometry”, Wylie J. Doods, Arch. Intern. Med., Vol. 136, May 1976; “A Method for Continuous Monitoring of Upper Oesophageal Sphincter Pressure”, Kahrilas, P., J. Dent, W. Dodds, W. Hogan, R. Arndorfer, Dig. Dis. Sci. 32:121-128, 1987 (Medline); “Modern Solid State Computerized Manometry of the Pharyngoesophageal Segment”, J. A. Castell, MS and D. O. Castell, MD, Dysphagia 8:270-275, 1993.

The present invention comprises a solution to the problems we know of today in registration of pressure profiles during swallowing.

### SUMMARY OF THE INVENTION

The present invention aims to provide a solution to the problems discussed above. This object is achieved through a system, device and method as defined in the appended claims.

Briefly, the system and device of the present invention comprises a manometric catheter having a first lumen formed with a window in the region of the pharynx and connected to a pressure sensor in an open system for perfusion manometry of the pharyngeal activity, and a second lumen mouthing in a water-filled cylindrical balloon and connected to a pressure sensor in a closed system for simultaneous and continuous registration of pressure variations in the UES.

In a preferred embodiment, the pressure profile recording system of the present invention comprises a first pressure sensor by which pressure variations in the pharyngeal muscle are continuously recordable upon swallowing, and a second pressure sensor by which pressure variations in the upper esophageal sphincter are simultaneously recordable, and a processor unit controlled to display timely correlated pressure profiles of the swallowing activities in the pharyngeal and cricopharyngeal muscles, respectively, derived from pressure data recorded by the first and second pressure sensors and obtained during a normal swallowing act, wherein pressures are collected by a manometric catheter insertable into the esophagus, said manometric catheter comprising:
a first lumen associated with said first pressure sensor and communicating with a water supply by which the first lumen is continuously flushed with water that exits from a window formed through the lumen wall;
a second lumen associated with said second pressure sensor and connecting a water supply to the interior of an inelastic, elongate balloon which is supported about the catheter with a proximal end of the balloon located about 30-40 mm distally of said window. Significant features of the invention are that the balloon has a length from a proximal end to a distal end such that, as the window levels with the tongue bone (hyoid bone) i.e. the hypopharynx in the inserted position of the manometric catheter, the balloon reaches from proximally of the upper esophageal sphincter in rest and distally past the upper esophageal sphincter with its distal end, and that the balloon is filled with water to one atmosphere internal pressure and as such insertable into the esophagus, as the result of which the second pressure sensor continuously registers absolute pressures from the upper esophageal sphincter during a normal swallowing act, notwithstanding a spontaneous vertical movement of the upper esophageal sphincter upon swallowing.

The method of the present invention comprises the simultaneous and continuous registration of pharyngeal and cricopharyngeal pressures. Timely correlated data from a swallowing act performed by the patient may thus be achieved, and pre-sented graphically by the system. Deviations from normal pressures and synchronization found in healthy persons are indicative of a possible medical condition, the identification of which may be facilitated when based on the presented results of the pressure profile recording method of the present invention.

Preferably, a method is provided by which pressure profiles are continuously and simultaneously recordable from the pharynx and from the upper esophageal sphincter during a normal swallowing act, comprising the steps of
providing a manometric catheter having a first lumen which opens through a window formed in the lumen wall, as well as a second lumen mouthing inside an inelastic elongate balloon supported about the catheter with a proximal end of the balloon located about 30-40 mm distally of said window;
connecting the first lumen to a first pressure sensor and to a water supply, and connecting the second lumen to a second pressure sensor and to a water supply;
filling the balloon with water to one atmosphere internal pressure;
positioning the manometric catheter outside the esophagus with the window leveling with the hyoid bone, located through palpation;
calibrating in this position the pressure sensors;
positioning the manometric catheter inside the esophagus with the window located in the pharynx according to the external calibration;
reading the pressure data collected by the two pressure sensors during a swallowing act and displaying in a graph the temporal correlation of the activities in the pharyngeal and cricopharyngeal muscles upon swallowing.

In accordance herewith a manometric catheter is provided and arranged to be insertable into the esophagus, said manometric catheter comprising:
a first lumen communicating with a water supply by which the first lumen is flushed with water that exits from a window formed through the lumen wall;
a second lumen connecting a water supply to the interior of an inelastic, elongate balloon which is supported about the catheter with a proximal end of the balloon located about 30-40 mm distally of said window, the manometric catheter characterized in that the balloon having a length from a proximal end to a distal end such that, as the window levels with the tongue bone (hyoid bone) i.e. the hypopharynx in the inserted position of the manometric catheter, the balloon reaches from proximally of the upper esophageal sphincter in rest and distally past the upper esophageal sphincter with its distal end, and in that the balloon is filled with water to one atmosphere internal pressure and as such insertable into the esophagus, as the result of which absolute pressures from the upper esophageal sphincter are recordable during a normal swallowing act, notwithstanding a spontaneous vertical movement of the upper esophageal sphincter upon swallowing.

Further embodiments of the invention in the above aspects are defined through the subordinated claims.

### DRAWINGS

The invention is more fully described below with reference to the accompanying drawings, wherein

FIG. 1 is a diagram showing a system set up for performing dual and simultaneous registration of pharyngeal and cricopharyngeal pressures;

FIG. 2 is a diagrammatic sectional view of a manometric catheter used in the system of FIG. 1;

FIG. 3 is a diagrammatic view showing the manometric catheter positioned in esophagus for dual and simultaneous registration of pharyngeal and cricopharyngeal pressures;

FIG. 4 is a diagram showing, for a healthy person, the typical pressure variations in pharyngeal region (upper graph) and UES region (lower graph) upon swallowing;

FIG. 5 is a diagram similar to FIG. 3 showing the graphs of a person suffering from cricopharyngeal achalasi resulting in defective relaxation and irregular cricopharyngeal contractions;

FIG. 6 is a diagram similar to FIG. 4 showing the graphs of the same person after surgery (in this case myotomi of cricopharyngeal muscle), and

FIG. 7 is a schematic registration curve in 28 healthy volunteers with subjectively normal swallowing activity (mean values and spread).

### DETAILED DESCRIPTION

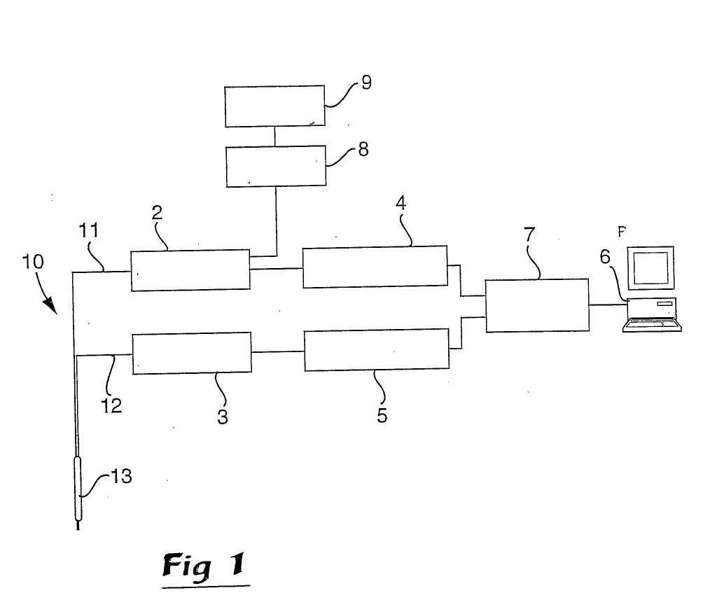

With reference to FIG. 1, a system 1 for performing dual and simultaneous registration of pharyngeal and cricopharyngeal pressures comprises a manometric catheter 10 having a first lumen formed with a window in the region of the pharynx and connected to a first pressure sensor 2 in an open system for perfusion manometry of the pharyngeal activity, and a second lumen mouthing in a water-filled balloon and connected to a second pressure sensor 3 in a closed system for simultaneous and continuous registration of pressure variations in the UES.

The pressure sensors 2, 3 are via preamplifiers 4, 5 and optical switches, respectively, connected to a processor unit or computer 6 through an A/D-converter 7. A flow regulating valve 8 controls the flow of sterilized water from a water supply 9 through the pressure sensor 4 and the first lumen of the manometric catheter 10.

The processor unit 6 is controlled by software to store pressure data received from both sensors 2 and 3, and to calculate and display pressure profiles showing the temporal correlation of activities in the pharyngeal and cricopharyngeal muscles upon swallowing. The pressure profiles are displayed on a computer screen and may be plotted on a printer (not shown) connected to the computer.

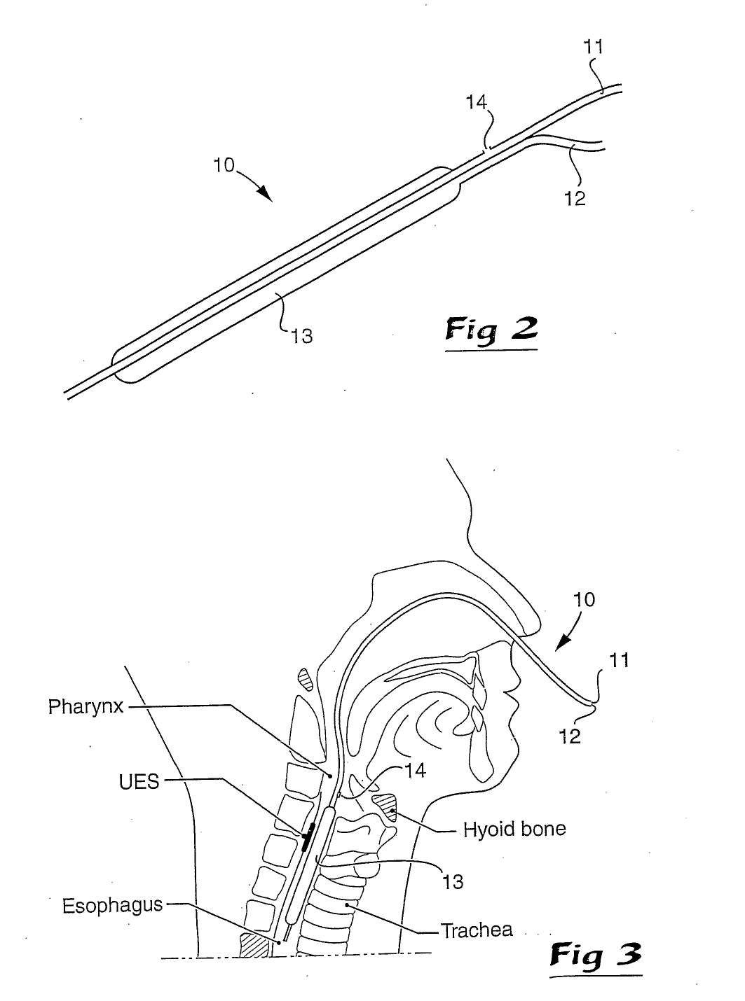

With reference to FIG. 2, the catheter 10 has a flexible body with two lumens, a first lumen 11 running from a proximal end of the body and mouthing in a distal end, and a second lumen 12 running from the proximal end and mouthing in an elongate balloon 13 surrounding the catheter body in a distal region of the catheter 10. The first lumen 11 communicates with a supply of sterilized water 9 and is during operation continuously flushed with water via the flow regulation valve 8. The water flow amounts to approximately 0.5 ml per minute, which exits through a window 14 formed through the wall of the first lumen 11 at a distance proximally from the balloon 13. The window 14 may advantageously be situated at a distance of approximately 30-40 mm from the proximal end of the balloon 13. In use, as illustrated in FIG. 3, the catheter 10 is inserted into esophagus to a depth that locates the window 14 in the region of the hyoid bone (tongue bone) which will correlate to the lower part of the hypopharynx. The water flow through the first lumen 11 passes the pressure sensor 2 which monitors the pressure and records a change in pressure when the window is occluded by the pharynx as the muscles are contracted in the initiating activity of a swallowing act. The registration of the pharynx activity by pressure sensor 2 is not necessarily a registration of absolute pressure, but rather a recording of a contraction in the pharyngeal muscles for evaluation of a temporal correlation with the registered activity in the UES.

In use, the balloon 13 is filled with water through the second lumen 12. The second lumen 12 communicates the water volume in the balloon 13 with the pressure sensor 3 in a closed system for registration of pressure variations in the UES. The balloon 13 preferably has a length of approximately 80-100 mm, preferably about 100 mm, and a continuous diameter between its proximal and distal ends preferably about 4 mm. In position for registration, wherein the window 14 of the first lumen 11 is located in the region of the hypopharynx, the proximal or upper portion of the balloon is located in the region of the UES high enough not to be dislocated despite any elevation of the UES that will normally occur in the swallowing act. Thus, the balloon 13 continuously records the activity in the higher pressure zone of the upper esophageal sphincter during the entire swallowing act, notwithstanding the normal and spontaneous vertical movements of the sphincter during swallowing.

The balloon 13 is an element having an inelastic wall, preferably made of polyethylene that is treated with respect to its molecular structure for increased strength and minimized stretching. An example of a suitable material for production of the balloon 13 is the Polyethylene MT, which can be found in balloon dilatation catheters provided by Medi-tech Inc., Massachusetts, USA.

Medium density polyethylene or equivalent synthetic material may likewise advantageously be used for the catheter body having two lumens. An example of commercially available catheters with two lumens is the dilatation catheters sold by Medi-tech Inc.

Essentially, the balloon 13 is made of an inelastic material and the manometric catheter system disclosed herein is characterized by a low yield or compliance, such as in the order of 0.2-0.4 microliter per mmHg within a maximum inflation pressure of up to approximately 6 atmospheres. The pressure gradient is 4000 mmHg/sec (40 Hz) at increase, and 2000 mmHg/sec (20 Hz) at decrease. The system's sensitivity to a change in temperature is negligible with regard to the perfusion system and “balloon system”, if care is taken not to “overfill” the balloon before calibration. “Intra-balloon pressure” must not be higher than 1 atmosphere upon closing of the system due to the highly non-distensible wall of the balloon. A small rise/fall in temperature will otherwise cause an influence on results. The system's sensitivity to altitude is 0.5 mmHg/cm. Between impression and detected pressure, the linearity is 1.0.

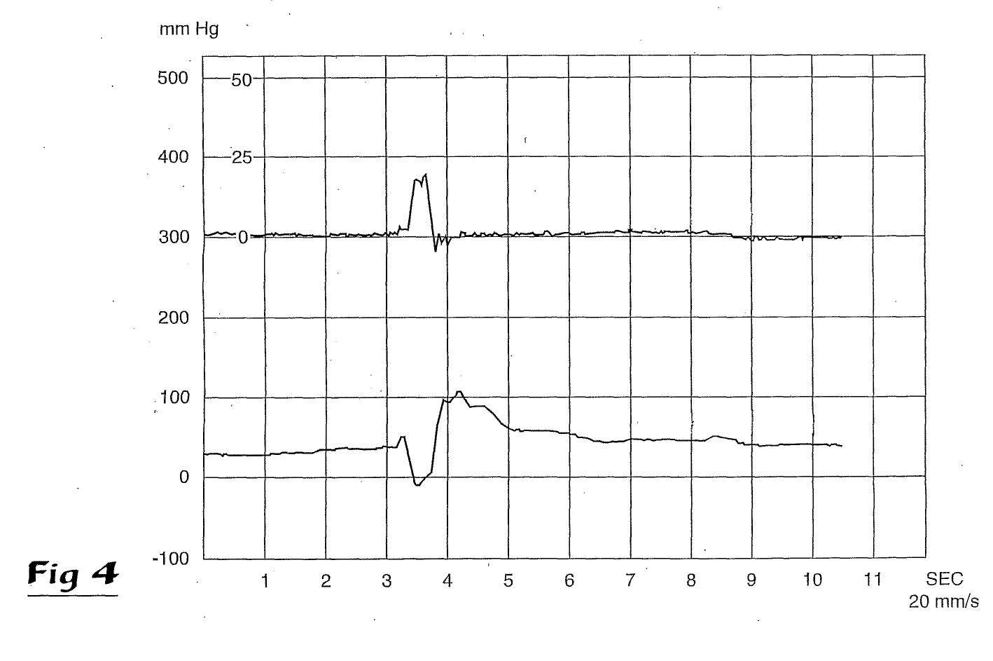

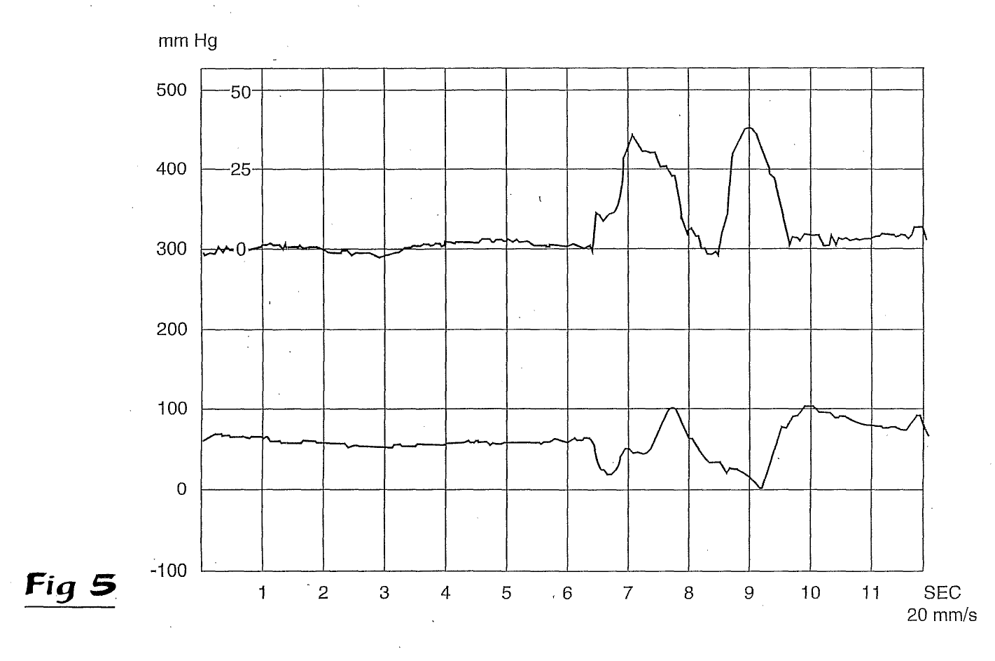

The pressure sensors 2,3 that were used in connection with the manometric catheter explained above to produce the graphs of FIGS. 4-7 are transducers of a laminar flow design having a pressure sensing diaphragm and microchip with resistors that are processed into the diaphragm. Suitable sensors are available, e.g., from Peter von Berg Medizintechnik GmbH, Kirchseeon/Eglharting, Germany.

Naturally, the referenced catheter material and transducers are to be understood merely as non-limiting examples that are readily available from the mentioned producers—equipment of other manufacture will serve equally well as long as the listed characteristics are essentially fulfilled.

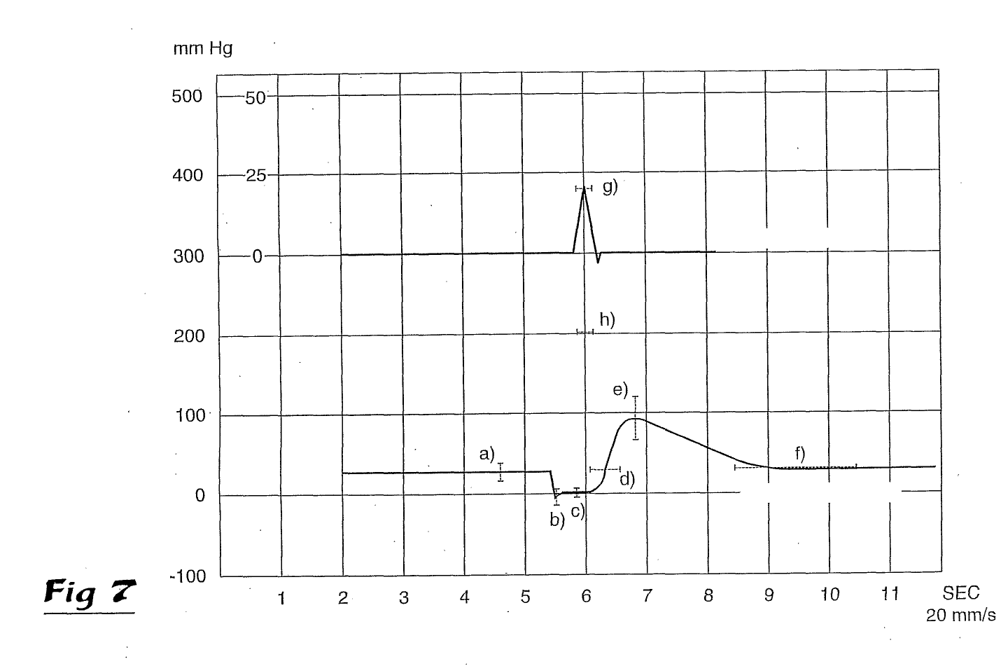

A listing of normal clinical values recorded from a reference group of 28 healthy individuals with subjectively normal swallowing activity (mean values+/−spread) is given below as an introduction to the following explanation of the registration procedure. See also FIG. 7 of the drawings.

| | | |
|---|---|---|
| a) | Normal pressure | 31.0 mmHg, +−10.0; |
| | in cricopharyngeus at rest | |
| b) | minimum pressure in | −3.3 mmHg +− 4.6 mm; |
| | relaxation phaseC | |
| c) | degree of relaxation | 1.0 +− 0.15 (decrease of |
| | | pressure in relaxation |
| | | phase/pressure at rest) |
| d) | relaxation time | 0.9 sec +− 0.3; |
| e) | maximum pressure at | 89.0 mmHg +− 29.0; |
| | contraction of UES | |
| f) | total time of a swallowing activity | 3.9 sec +− 1.2; |
| g) | maximum pressure at | 20.0 mmHg +− 9.5. |
| | contraction of pharynx | |
| h) | coordination | 0.7 +− 0.16 |
| | (pharynx max. amp./relax. UES) | |

Preparing the system and manometric catheter 10 described herein for a registration procedure involves calibration of the pressure sensors 2 and 3. Using a mercury manometer with rubber bladder or similar device as guide, the pressure sensor 3 detecting pressure variations in UES is calibrated for registering, in the computer 6, pressures ranging from 0 to 300 mmHg. Adjustment of the measurement range displayed on the computer may be achieved through controls provided on the associated preamplifier, for correspondence between the values displayed and recorded by the computer and the pressures applied to the system and detected by the balloon 13 and the pressure sensor 3. The pressure sensor 2, detecting the activity in pharynx, is calibrated for a measurement range of 0 to 50 mmHg using the same procedure.

Next, the balloon 13 is filled with water to hold atmospheric pressure, making sure that the closed system is completely free from gas bubbles/air pockets.

The system is then calibrated with respect to altitude, or height above sea level. The manometric catheter 10 is placed outside the patient such that the window 14 is positioned at the same level as the tongue-bone (hyoid bone), which may be located through palpation. At this level, the computer is adjusted to indicate a zero pressure.

The manometric catheter 10 is then inserted into esophagus through the patient's nose, using a guide wire, until the balloon 13 is positioned well below the UES.

From this position the catheter 10, first flushed with water through the open system, is slowly withdrawn to the zero pressure level where the window 14 will be located in the hypopharynx. The balloon is now located in the UES and the pressure at rest in the UES is continuously registered by the closed system. Correct position of the device is also verified by the obvious appearance of a rise in balloon pressure from below zero intrathoracic pressure to the resting-pressure level in the UES, which is well above zero. From this position or check point, the manometric catheter 10 is further withdrawn for a length of approximately 10 mm, this way securing registration of pressure variations in the UES despite the notorious elevation of the high-pressure zone occurring in the swallowing act. Now correctly positioned, the manometric catheter 10 is fixated at the entrance, e.g. by applying a surgical tape to the patient's nose.

The patient is then given water to swallow, at volumes of approximately 5-10 ml drawn through a suction pipe, e.g. Pressure variations in pharynx and UES are registered during repeated swallowing, upon direction of the investigator, until a reproducible pattern is achieved. As stated above, the registration of the pharynx activity through pressure sensor 2 of the open system is not necessarily a registration of absolute pressure, but rather a detection of a contraction in the pharyngeal muscles for evaluation of a temporal correlation with the true registered pressures in the cricopharyngeal muscle (UES). The balloon 13 and pressure sensor 3 of the closed system continuously registers the activity in the high pressure zone (UES) during the entire swallowing act, notwithstanding the normal movements of the sphincter. In addition, the closed system registers pressure variations circumferentially about the balloon 13, this way compensating for the radial asymmetry of the pressure in the sphincter zone.

The readings are plotted in graphs, see FIGS. 4-6. The graphs show recorded pressures on the vertical scale, and time on the horizontal scale. In the graphs, the measurement ranges of sensors 2 and 3 are noted in parallel on the vertical axis. The upper curve shows the registered activity in pharynx during a swallowing act, and the lower curve the pressure variations in UES. The temporal correlation of relaxation and contraction in the two muscle-groups is readily apparent from the two curves.

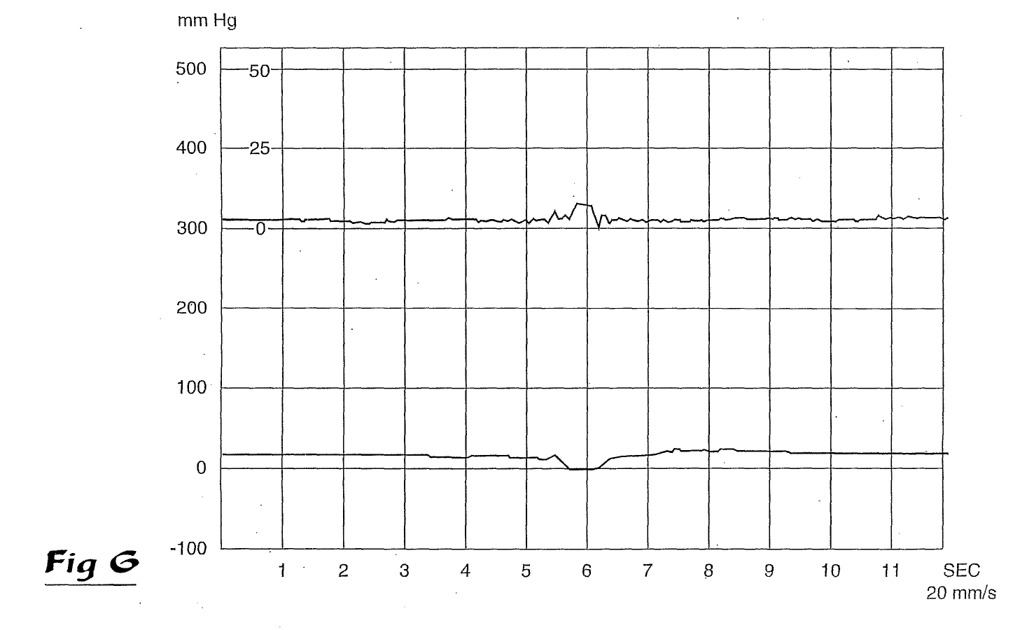

Thus, FIG. 4 is a graph typical for the recordings from a healthy person during swallowing. All parameters of clinical interest are within normal limits. FIG. 5 on the other hand is a graph produced from the recordings of a person suffering from swallowing disorder, in this case a cricopharyngeal bar confirmed by a pathological X-ray investigation. The registration graph shows increased resting pressure in the UES, double swallows, incomplete UES relaxation, increased pharyngeal wave amplitude and duration and non-coordination of muscular activity. Typical findings for the diagnosis cricopharyngeal achalasi. FIG. 6 shows the recordings of the same person (FIG. 5) now free from symptoms after operation—in this case myotomidivision of the UES muscle. Graph shows lowered UES resting pressure, single swallow activity, complete relaxation of pressure in sphincter region and low and coordinated pharyngeal activity. No contraction seen in UES area (remaining resting pressure in UES due to passive force of larynx to cervical spine).

As understood from the above, the pressure profile recording system manages to register continuously the pressure at rest as well as rapid pressure variations in UES, and registers dynamically as the UES moves vertically during the swallowing act. The structure and profile of the manometric catheter 10 provides maximum contact circumferentially with surrounding muscles and tissue for registration of pressure conditions in the high-pressure zone, from where the true maximum pressures are obtained by the inelastic balloon which is calibrated to atmospheric pressure at the operating level. Securing the correct operating level is accomplished by using the perfusion system to locate the manometric catheter relative to the patient, firstly outside the patient during calibration and secondly within the esophagus during registration.

1. A pressure profile recording system, comprising a first pressure sensor (2) by which pressure variations in the pharyngeal muscle are continuously recordable upon swallowing, and a second pressure sensor (3) by which pressure variations in the upper esophageal sphincter (UES) are simultaneously recordable, and a processor unit (6) controlled to display timely correlated pressure profiles of the swallowing activities in the pharyngeal and cricopharyngeal muscles, respectively, derived from pressure data recorded by the first and second pressure sensors (2; 3) and obtained during a normal swallowing act, wherein pressures are collected by a manometric catheter (10) insertable into the esophagus, said manometric catheter comprising:
a first lumen (11) associated with said first pressure sensor (2) and communicating with a water supply by which the first lumen is continuously flushed with water that exits from a window (14) formed through the lumen wall;
a second lumen (12) associated with said second pressure sensor (3) and connecting a water supply to the interior of an inelastic, elongate balloon (13) which is supported about the catheter with a proximal end of the balloon (13) located about 30-40 mm distally of said window (14), characterized in that the balloon (13) having a length from a proximal end to a distal end such that, as the window (14) levels with the tongue bone (hyoid bone), i.e. the hypopharynx, in the inserted position of the manometric catheter (10), the balloon (13) reaches from proximally of the upper esophageal sphincter in rest and distally past the upper esophageal sphincter with its distal end, and in that the balloon (13) is filled with water to one atmosphere internal pressure and as such insertable into the esophagus, as the result of which the second pressure sensor (3) continuously registers absolute pressures from the upper esophageal sphincter during a normal swallowing act, notwithstanding a spontaneous vertical movement of the upper esophageal sphincter upon swallowing.

2. The system of claim 1, characterized in that the balloon (13) having a length of 80-100 mm, preferably 100 mm.

3. The system of claim 1, wherein the first pressure sensor (2) is calibrated for measurements within a range of 0-50 mmHg, and the second pressure sensor (3) is calibrated for measurements within a range of 0-300 mmHg.

4. A manometric catheter (10) insertable into the esophagus, said manometric catheter comprising:
a first lumen (11) communicating with a water supply by which the first lumen is flushed with water that exits from a window (14) formed through the lumen wall;
a second lumen (12) connecting a water supply to the interior of an inelastic, elongate balloon (13) which is supported about the catheter with a proximal end of the balloon (13) located about 30-40 mm distally of said window (14), characterized in that the balloon (13) having a length from a proximal end to a distal end such that, as the window (14) levels with the tongue bone (hyoid bone), i.e. the hypopharynx, in the inserted position of the manometric catheter (10), the balloon (13) reaches from proximally of the upper esophageal sphincter in rest and distally past the upper esophageal sphincter with its distal end, and in that the balloon (13) is filled with water to one atmosphere internal pressure and as such insertable into the esophagus, as the result of which absolute pressures from the upper esophageal sphincter are recordable during a normal swallowing act, notwithstanding a spontaneous vertical movement of the upper esophageal sphincter upon swallowing.

5. The manometric catheter (10) of claim 4, wherein the balloon (13) has a length of 80-100 mm, preferably 100 mm.

6. The manometric catheter (10) of claim 5, wherein the balloon (13) has a length of approximately 100 mm and a diameter of approximately 4 mm.

7. The manometric catheter (10) of claim 4, wherein the balloon (13) has an inelastic synthetic material wall, preferably made of polyethylene.

8. A method by which pressure profiles are continuously and simultaneously recordable from the pharynx and from the upper esophageal sphincter (UES) during a normal swallowing act, comprising the steps of
providing a manometric catheter (10) having a first lumen (11) which opens through a window (14) formed in the lumen wall, as well as a second lumen (12) mouthing inside an inelastic elongate balloon (13) supported about the catheter with a proximal end of the balloon (13) located about 30-40 mm distally of said window (14);
connecting the first lumen (11) to a first pressure sensor (2) and to a water supply (9), and connecting the second lumen (12) to a second pressure sensor (3) and to a water supply;
filling the balloon (13) with water to one atmosphere internal pressure;
positioning the manometric catheter outside the esophagus with the window (14) leveling with the hyoid bone, located through palpation;
calibrating in this position the pressure sensors (2,3);
positioning the manometric catheter inside the esophagus with the window (14) located in the hypopharynx according to the external calibration;
reading the pressure data collected by the two sensors (2; 3) during a swallowing act and displaying in a graph the temporal correlation of the activities in the pharyngeal and cricopharyngeal muscles upon swallowing.

9. The method of claim 8, wherein the first pressure sensor (2) is calibrated for recording pressures within a range of 0-50 mmHg, and the second pressure sensor (3) is calibrated for recording pressures within a range of 0-300 mmHg.

10. The manometric catheter (10) of claim 5, wherein the balloon (13) has an inelastic synthetic material wall, preferably made of polyethylene.

11. The manometric catheter (10) of claim 6, wherein the balloon (13) has an inelastic synthetic material wall, preferably made of polyethylene.
